(19) United States
(12) Patent Application Publication
(10) Pub. No.: US 2019/0038842 A1
(43) Pub. Date: Feb. 7, 2019

(54) A HYPOCYCLOID END-OF-CONTENT MECHANISM

(71) Applicant: Novo Nordisk A/S, Bagsvaerd (DK)

(72) Inventors: Simon Munch Pedersen, Copenhagen N (DK); Simon Roervig, Copenhagen OE (DK)

(21) Appl. No.: 16/074,682

(22) PCT Filed: Feb. 2, 2017
(86) PCT No.: PCT/EP2017/052198

§ 371 Date: Aug. 1, 2018

(30) Foreign Application Priority Data
Feb. 2, 2016 (EP) 16153816.0

(51) Int. Cl. A61M 5/315

(52) U.S. Cl. CPC A61M 5/31541; A61M 2005/2013; A61M 5/31591; A61M 5/31553

(57) ABSTRACT

The invention relates to a torsion spring driven injection device for delivering individually set doses of a liquid drug wherein a torsion spring is strained by rotation of a rotatable dose setting element and released by axial movement of a clutch which couples the dose setting element with the drive tube and the drive tube with a piston rod guide driving the piston rod. When straining the torsion spring the clutch is decoupled from a piston rod guide such that the piston rod guide is able to rotate independently and coupled to the dose setting element. During dose expelling the clutch is moved into engagement with the piston rod guide and out of engagement with the dose setting element such that the piston rod guide rotates with the clutch under influence of the torsion spring. The invention further relates to a non-axial movable End-of-Content mechanism preventing the user from setting a dose larger than the content of the injectable liquid drug remaining in the cartridge of the injection device.

### THE TECHNICAL FIELD OF THE INVENTION

The present invention relates to a torsion spring driven injection device for delivering individually set doses of a liquid drug. The present invention especially relates to the mechanism by which the torque of the torsion spring is released and transferred to an axial movement of the piston rod.

The present invention further relates to a hypocycloid End-of-Content mechanism for a medical injection device for injecting a liquid drug and especially to a hypocycloid End-of-Content mechanism for a pre-filled torsion spring driven injection device.

### DESCRIPTION OF RELATED ART

International patent application WO 2014/060369 discloses an example of a torsion spring driven injection device for delivering individual set doses of a liquid drug. In this known injection device a torsion spring is strained when setting a dose and released to drive a piston rod forward to expel the set dose. The torque held in the torsion spring is released by moving a distal needle shield in the proximal direction. An injection device wherein the torque is released by the axial movement of the needle shield is often referred to as shield triggered injection devices.

In the torsion spring driven injection device disclosed in WO 2014/060369, an axially movable clutch transfers the release force from the distal needle shield to the drive arrangement such that the torque of the torsion spring is released when the needle shield is pressed against the skin of the user during injection. Henceforth, in order to release the torque stored in the torsion spring the user presses the needle shield against the skin during injection such that the needle shield moves axially in the proximal direction. This axial movement of the needle shield is transferred to a corresponding axial movement of the clutch which again transfers its axial movement to an axial movement of the ratchet element.

The axial movement of the clutch removes the clutch from its engagement with the housing and engages the clutch with the drive tube whereas the axial movement of the ratchet element releases the ratchet element from the drive tube thus making the drive tube able to be rotated by the torsion spring and the clutch to follow the rotation of the drive tube. Since the drive tube is axially fixed due to the torsion spring, the ratchet element needs to move axially.

Since the EoC (End-of-Content) disc is located on the outer surface of the clutch as disclosed in FIG. 1 in WO 2014/060369, this disc-shaped EoC element must follow the axial movement of the clutch during release of the torque. This axial movement of the disc-shaped EoC element must be performed without altering the accumulated doses counted by the Disc-shaped EoC element at the moment of axial translation.

Both the release and the counting of the doses thus require axial translation of a significant number of individual parts inside the housing of the torsion spring driven injection device including the disc-shaped EoC element.

In this, and many other, injection devices for injecting an adjustable amount of a liquid drug the user usually rotates a button in order to set the adjustable size of the dose to be injected. When the injection device is the elongated pen-shaped type, this does setting button is usually provided at a proximal end on the injection device. Such injection devices holds a cartridge containing a specific amount of liquid drug and is usually equipped with a mechanism which secures that a user cannot set a dose size which exceeds the injectable amount remaining in cartridge at any time.

In mechanical injection devices this mechanism is usually a counter which is moved to a new position whenever a dose is set but maintained in this new position when the dose is injected. The position of the counter is thus an expression of the accumulated doses set by the user. The movement of the counter is then restricted in accordance with the initial injectable quantum in the cartridge such that the counter is blocked in its movement when the accumulated doses set equals the initial injectable quantum in the cartridge.

Such mechanism is often referred to as an End-of-Content (EoC) mechanism and a very simple example is provided in U.S. Pat. No. 4,973,318. In this injection device the counter nut is formed integral with the dose setting button and is rotated up the threaded piston rod when a dose is set. When the set dose is injected, the counter nut is maintained in its relatively position on the thread of the piston rod as the dose setting button and the piston rod is moved axially forward. The length of the thread correlates to the initial injectable quantum of liquid drug in the cartridge and once the counter nut reaches the end of the thread no further dose can be set.

However, in this injection device the axial distance the injection button is moved during injection corresponds to the axial distance that the piston rod is moved forward inside the cartridge.

More modern injection devices has a gearing mechanism such that the piston rod can be moved a different length than the injection button is moved. An End-of-Content mechanism for such modern injection devices is disclosed in US RE41.956.

FIG. 3 of US RE41.956 discloses an embodiment in which a counter nut is moved up a helical track on a driver whenever a dose setting member is rotated. During injection, the counter nut is maintained in its relative position in the helical track such that the position of the counter nut in the helical track at any time is an expression of the accumulated doses set by the user. The length of the helical track correlates to the initial injectable quantum of liquid drug in the cartridge and once the counter nut reaches the end of the helical track, the dose setting member cannot be rotated further thus a dose larger than what corresponds to the length of the helical track cannot be set.

FIG. 2 of US RE41.956 discloses a different embodiment wherein the End-of-Content mechanism is non-axial working. Here the driver is provided with a spiral track and the dose setting member is provided with a track follower engaging the track. The track and the track follower is rotated relatively to each other during dose setting but maintained in a relatively fixed position during injection. Once the spiral track ends, the track follower and thus the dose setting member cannot be moved further. However, since the length of the spiral track has to correlate to the initial injectable quantum of drug in the cartridge, the driver need to have a rather large diameter which disqualifies the use of this type of EoC mechanism in pen shaped injection devices.

An injection device similar to the one disclosed in FIG. 3 of US RE41.956 is disclosed in WO 2013/170392. This injection device has a dose setting button which travels axially both during dose setting and during expelling of the set dose. Internally this dose setting button is provided with an End-of-Content mechanism which thus also travels axially both during dose setting and during expelling of the set dose. The End-of-Content mechanism disclosed in this document is based on a planetary gear mechanism having a planetary element that rotates around its own axis by a rotation of an outer element. After the planetary element has rotated several times around its own axis the planetary element encounters a stop hindering further dose setting.

A similar rotational stop mechanism limiting the number of revolutions of a shaft is disclosed in U.S. Pat. No. 3,411,366.

A different End-of-Content mechanism is disclosed in EP 1,861,141. In this EoC mechanism a first rotatable element rotates a second rotatable element one increment for each full rotation of the first element. A mechanism is provided which moves the second element axially in relation to the first element such that the two elements only engages and rotate together once for each full rotation of the first element. Once the second element has been rotated a specific and predetermined number of times the second element is arrested by a stop means and thus prevents both the second element and the first element from being rotated further. However, the axial movement of the second rotatable element in and out of its engagement with the first element requires some axial space inside the injection device.

In the recent years automatic spring driven injection devices have become very popular. These injection devices has a spring, often a torsion spring, which is strained during dose setting and released to drive a piston rod forward during injection. Since the spring provides the force to drive the injection there is no need for the user to push an injection button back into the housing of the injection device during injection. These new injection devices therefore have no part which grows out from the housing during dose setting in order for a user to push the same part back into the housing during dose injection. As a result these new automatic injection devices have the same length all the time.

An example of an End-of-Content mechanism for such automatic injection device is disclosed in WO2007/017052. Here a helically movable counter nut is screwed up the thread on the threaded piston rod when a dose is set and maintained in its relative position during dose injection. Once the counter nut reaches the end of the thread on the piston rod, the counter nut prevents the dose setting member from being rotated any further which thereby prevents that a further dose in being set. The length of the thread on the piston rod correlates to the initial injectable amount of liquid drug in the cartridge such that the counter nut reaches the end of the track when the initial injectable quantum has been repetitive set.

A drawback for all these known End-of-Content mechanism is that they require either a substantial clear axial length of the injection device due to the axial working element or a relatively large diameter in order to carry the spiral track as in US RE41.956 FIG. 2.

A torsion spring driven injection device having a hypocycloid geared End-of-Content mechanism is further described in WO 2014/117944 and a similar rotational stop mechanism limiting the number of revolutions of a shaft is disclosed in GB 862,641.

A further hypocycloid End-of-Dose mechanism for a powder inhaler is disclosed in U.S. Pat. No. 5,263,475. In this construction an indicator ring carrying a stop is rotated each time a dose is set and after a predetermined number of doses has been set and released, the stop enters into abutment with another stop thus preventing further dose setting.

### DESCRIPTION OF THE INVENTION

It is an object of the present invention to provide a torsion spring driven injection device for delivering individually set doses of a liquid drug wherein the release mechanism is made less cumbersome. It is especially an object to provide such torsion spring driven injection device requiring only a few parts and preferably only a single part to move axially in order to properly release the torque stored in the torsion spring.

It is further an object to provide an End-of-Dose mechanism wherein the disc-shaped EoC element is not required to move axially.

Accordingly, in one aspect, the present invention relates to a torsion spring driven injection device for delivering individually set doses of a liquid drug. The injection device comprises:
A housing formed around the mechanical parts making up the torsion spring engine. Such housing can be formed from one part or from a number of parts connected to each outer to form a housing structure.
A rotatable dose setting element (or ratchet element) axially fixated in the housing and by which a user is able to select the size of a dose to be injected. The rotatable dose setting element being rotatable in both rotational directions to respectively set and increase the dose size or to reduce an already set dose size.
A piston rod having an outer surface with an outer thread which thread extend helically in a longitudinal direction and which outer surface further is provided with a longitudinal extending engagement surface. Due to the presence of such longitudinal engagement surface, the outer surface of the piston rod has a non-circular shape.
A nut member having an inner thread mating with the outer thread of the piston rod thus making the nut member and the piston rod able to move helically in relation to each other.
A piston rod guide engaging the longitudinal extending engagement surface of the piston rod such that the piston rod is moved axially when the piston rod guide and the nut member are rotated relatively to each other, and wherein either the nut member or the piston rod guide is non-rotational engaged in the housing,
A drive tube which is axially fixated in the housing and engaging an axially movable clutch.
A torsion spring which is strained by rotation of the rotatable dose setting element and which torsion spring is encompassed between the housing and the drive tube such that a torque is build up in the torsion spring during rotation of the drive tube relatively to the housing during dose setting and which torque is released to rotate the drive tube during ejection,

Further, the clutch is axially movable in relation to the drive tube between a first position and a second position.

In the first position, the clutch is decoupled from the piston rod guide such that the piston rod guide is able to rotate independently of the clutch and the clutch is coupled with the rotatable dose setting element to rotate simultaneously with the rotatable dose setting element in order to set a dose.

In the second position, the clutch is decoupled from the rotatable dose setting element such that the clutch is able to rotate independently of the rotatable dose setting element under influence of the torsion spring and the clutch is coupled with the piston rod guide such that the piston rod guide rotates simultaneously with the clutch in order to drive the piston rod.

Besides being axially movable in relation to the drive tube, the clutch is rotational locked to the drive tube such that the clutch and the drive tube rotates in unison in a first rotational direction by rotation of the dose setting element when the clutch is in the first position thereby straining the torsion spring to build up torque, and the clutch and the drive tube rotates in unison in a second opposite rotational direction by the torsion spring when the clutch is in the second position thereby releasing the torque of the torsion spring.

In the above described torsion spring driven injection device, both the dose setting element (the ratchet element) and the drive tube are axially fixated such that the only and single part moving axially during dose injection is the clutch. The axial movement of the clutch can be performed in many different ways e.g. either by axial movement of a needle shield or by a sliding button provided on the housing.

A further benefit for the above described construction is that the torsion spring engine can be manufactured as one component for later incorporation into the torsion spring driven injection device. In one example, the torsion spring engine thus comprises:
the spring base attachable to the housing,
the dose setting element (i.e. the ratchet element) with or without the dose dial,
the torsion spring,
the drive tube,
the clutch and preferably a compression spring encompassed between the clutch, and drive tube.

After the torsion spring engine has been assembled it is carried by the spring base and can be loaded into the housing and attached to the housing by attaching the spring base to the housing. Further, the nut member can be secured to the housing in a non-rotational manner.

The spring base which also has the torsion spring attachment can be a separate part interconnected with the housing or it can alternatively be moulded unitarily with the housing.

In order to better distinct between the first position and the second position of the clutch, a compression spring is provided between the drive tube and the clutch urging the drive tube and the clutch in opposite axial directions.

In order to count the doses set and ejected an End-of-Content mechanism is provided. This EoC mechanism has no components working in the axial or longitudinal direction of the torsion spring driven injection device which makes it possible to shorten the total length of the torsion spring driven injection device. An example of such non-axially working End-of-Content mechanism is given in WO 2014/117944 which in details explains the working principle of a similar hypocycloid End-of-Content system.

In one example the ratchet element is provided with an annular bearing flange which rotatably carries a rotatable disc-shaped EoC element. The annular bearing flange can either be cylindrical such that the disc-shaped EoC element performs a circular rolling movement on the flange or it can be eccentric such that the disc-shaped EoC element performs a hypocycloid movement.

Further, the disc-shaped EoC element can be provided with one or more axially extending protrusions one of which are preferably rotated an angle in a track associated with the housing during dose setting. This peripheral track provided in the housing or in the spring base coupled to the housing is preferably substantially smaller than one full revolution i.e. 360 degrees meaning that the path described by the rotation of the axially extending protrusion around the centre axis is not a continually closed path.

In one embodiment, the EoC mechanism is a hypocycloid End-of-Content mechanism wherein the disc-shaped EoC element rotates less than a full circle. In a second embodiment, the spring base is provided with a peripheral track guiding at least one of the protrusions provided on the disc-shaped EoC element and in a third embodiment an additional EoC hypocycloid gear ring is provided.

In the first embodiment of the non-axially working End-of-Content system, the torsion spring driven injection device further comprises:
A rotatable dose setting element rotatable around a first axis, and
A cycloid disc-shaped EoC element rotatable around a second axis which second axis is dislocated in relation to the first axis.

Further, arresting means are provided for stopping the rotation of the cycloid disc in a predetermined position.

These arresting means preferably comprises an axially extending protrusion provided on the cycloid disc and a stopping position provided in a guiding track in the housing, preferably in the spring base connected to the housing. The peripheral length of the guiding track is substantially less than one full rotation. Preferably, the peripheral length of the guiding track is around one half of a full rotation.

When rotating the dose setting element in a first rotational direction, the disc-shaped EoC element is rotated one increment in the opposite rotational direction for each full rotation of the dose setting element. The position of the axial protrusion provided on the disc-shaped EoC element in the guiding track is thus an expression on the number of rotations of the dose setting element and thus an expression of the set and previously expelled doses. The guiding track engaged by the axial protrusion is made with a length limiting the movement of the protrusion to the number of possible doses.

If the torsion spring driven injection device in one example is prefilled with 300 International Units (I.U.) of insulin and one rotation equals a setting of e.g. 24 I.U., the length of the track must be able to stop further rotation of the axial protrusion after 12.5 full rotations of the dose setting element such that the dose setting element is only able to rotate 12.5 times 360 degrees.

In a second embodiment of the non-axially working End-of-Content system, the torsion spring driven injection device further comprises:
A rotatable dose setting element rotatable around a first axis, and
A disc-shaped EoC element having a first protrusion guided in a first track and a second protrusion guided in a second track.

Further, the second track is provided with at least one curled part introducing at least one oscillating radial movement to the disc-shaped EoC element for each full rotation of the dose setting element, and arresting means for stopping the rotation of the cycloid disc in a predetermined position.

In the second embodiment, the stopping means is also a guiding track having a limited length of less than one full rotation. The first protrusion is guided in this stop track preferably provided in the spring base as part of the housing whereas the second protrusion is guided in a second track provided in the dose setting element.

For each full rotation of the dose setting element, the second protrusion is guided through the curled part of the second track which introduces an oscillating movement in the disc-shaped EoC element. The disc-shaped EoC element is for this purpose provided with an oval opening and guided on a cylindrical annual bearing of the dose setting element such that the dose element is allowed to rotate and the disc-shaped EoC element is allowed to oscillate once for each full rotation of the dose setting element. This oscillating movement moves the first protrusion one increment in the first track and once the injectable content of the cartridge has been set, the first protrusion encounters the end of the first track and thus prevents further dose setting.

Referring to the first example, the first track needs to accommodate 12.5 incremental movements of the first protrusion thus allowing the dose setting element to rotate 12.5 times 360 degrees.

In a third embodiment of the non-axially working End-of-Content system, the torsion spring driven injection device further comprises:
A rotatable dose setting element rotatable around a first axis, and
A ring-shaped EoC gear element rotatable around a second axis which second axis is dislocated in relation to the first axis, whereby rotation of the ring-shaped EoC gear element is transformed to a rotation of a cycloid disc-shaped EoC element.

Further, arresting means are provided for stopping the rotation of the cycloid disc in a predetermined position.

As in the first embodiment rotation of the dose setting element induces a hypocycloid rotation of a ring-shaped EoC gear element in the opposite direction. This rotation of the ring-shaped EoC gear element further rotates the disc-shaped EoC element which is thereby rotated in the same rotational direction as the dose setting element however in a gearing ratio defined by the ring-shaped EoC gear element.

As in the first embodiment, the arresting means comprises an axially extending protrusion provided on the disc-shaped EoC element and a stopping position provided in a track in the housing and preferably in the spring base.

The operable length of the track provided in the spring base of the housing is decided by the gearing ratio of the ring-shaped EoC gear element and is set such that the axial protruding protrusion encounters the end of the track when the accumulated doses set by the dose setting element equals the injectable amount of liquid drug initially provided in the cartridge.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and connected to a hub to form a complete injection needle also often referred to as a “needle assembly” or “injection needle”. A needle cannula could however also be made from a polymeric material or a glass material. The hub also carries the connecting means for connecting the needle assembly to an injection apparatus and is usually moulded from a suitable thermoplastic material.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device or permanently connected to the injection device e.g. via a cartridge holder such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

“Scale drum” is meant to be a cylinder shaped element carrying indicia indicating the size of the selected dose to the user of the injection pen. The cylinder shaped element making up the scale drum can be either solid or hollow. “Indicia” is meant to incorporate any kind of printing or otherwise provided symbols e.g. engraved or adhered symbols. These symbols are preferably, but not exclusively, Arabian numbers from “0” to “9”. In a traditional injection pen configuration the indicia is viewable through a window provided in the housing.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism e.g. in the form of a button on, e.g. on the proximal end, of the injection device to release—fully or partially—the force accumulated in the spring when carrying out the injection.

The term “Permanently connected” as used in this description is intended to mean that the parts, which in this application is embodied as a cartridge and a housing structure, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show an exploded view of the injection device according to a first embodiment of the invention.

FIG. 2 show a cross sectional view of the spring engine of FIG. 1.

FIG. 3 show a side view of the ratchet element of FIGS. 1 and 2

FIG. 4 show an exploded view of the End-of-Content system of the injection device disclosed in FIG. 1.

FIG. 5 show a top view of the spring base of FIG. 4 viewed from a proximal position.

FIG. 6 show a perspective view of the cycloid disc-shaped EoC element disclosed in FIG. 4.

FIG. 7 show an exploded view of the injection device according to a second embodiment of the invention.

FIG. 8 show an exploded view of the End-of-Content system of the second embodiment shown in FIG. 7.

FIG. 9 show a side view of the ratchet element of FIG. 8.

FIG. 10 show a top view of the spring base of FIG. 8 viewed from a proximal position.

FIG. 11 show a view of the proximal end of the ratchet element of FIG. 8 as viewed from a distal position.

FIG. 12 show perspective view of the disc-shaped EoC element disclosed in FIG. 6.

FIG. 13 show an exploded view of the injection device according to a third embodiment of the invention.

FIG. 14 show an exploded view of the End-of-Content system of the third embodiment shown in FIG. 13.

FIG. 15 show a top view of the spring base of FIG. 14 viewed from a proximal position.

FIG. 16a-b show views of the disc-shaped EoC element disclosed in FIG. 14.

FIG. 17a-b show views of the cycloid gearing element of the FIG. 14.

The figures are schematic and simplified for clarity, and they just show details, which are essential to the understanding of the invention, while other details are left out. Throughout, the same reference numerals are used for identical or corresponding parts.

### Detailed Description of Embodiment

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

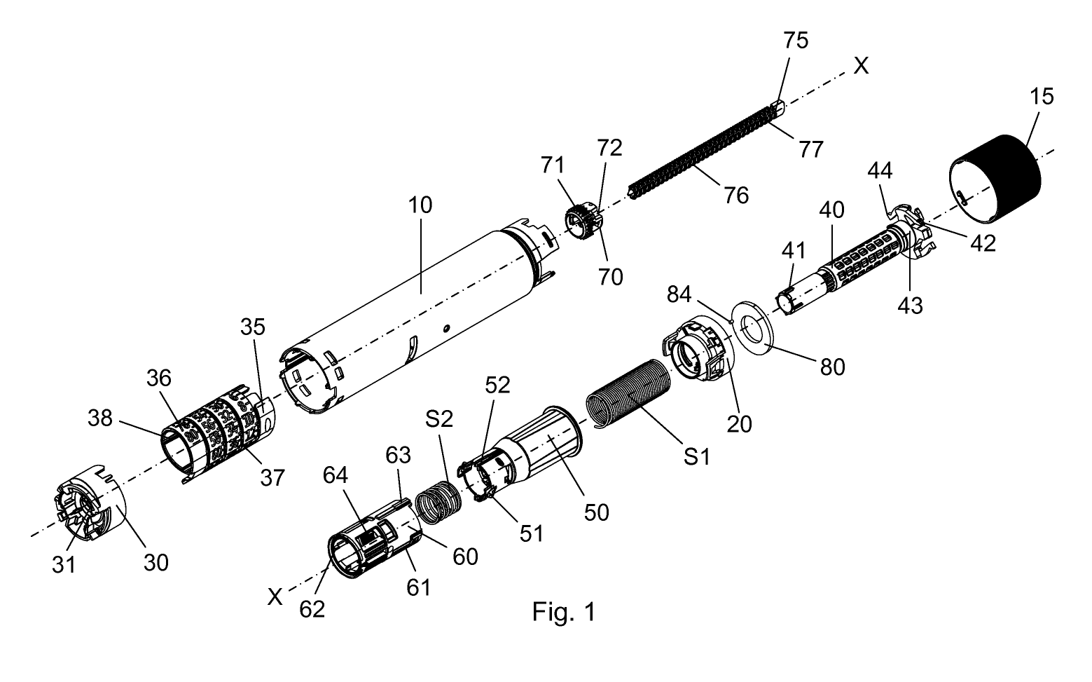

In that context it may be convenient to define that the term “distal end” in the appended figures is meant to refer to the end of the injection device which usually carries the injection needle whereas the term “proximal end” is meant to refer to the opposite end pointing away from the injection needle and usually carrying the dose dial button. Distal and proximal is meant to be along an axial orientation of the injection device along a virtual centre line marked “X” in FIG. 1.

### The Torsion Spring Engine

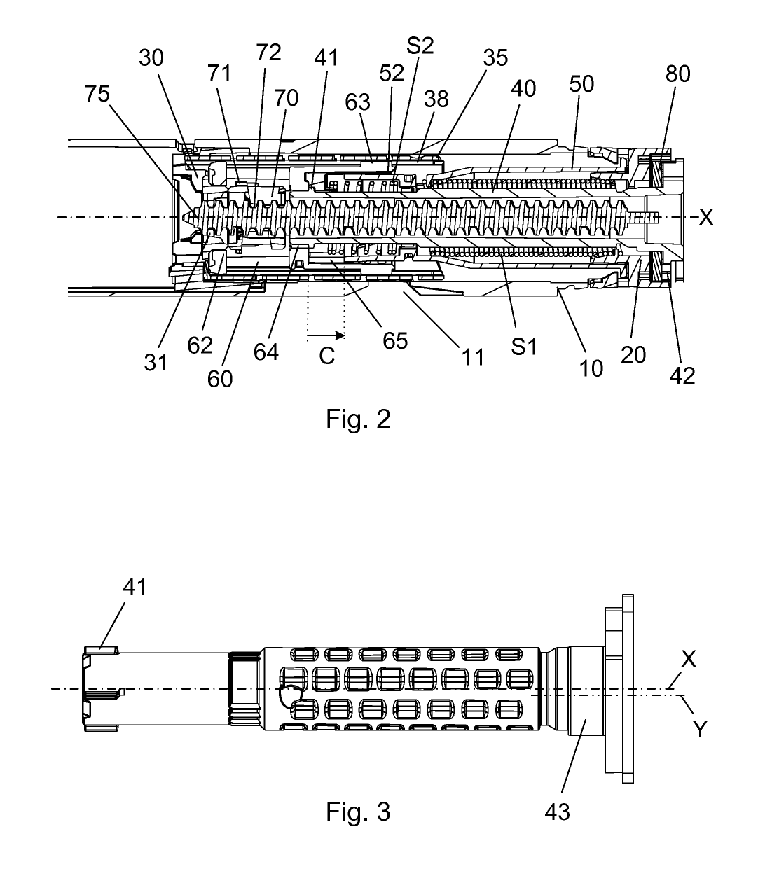

FIG. 1 and FIG. 2 disclose the torsion spring engine for a torsion spring driven medical injection device according a first embodiment of the invention. Such torsion spring driven medical injection device is usually an oblong pre-filled injection pen provided with an injection needle at the distal end.

By torsion spring engine is here meant the mechanism which secures the torsion spring and allows the torsion spring to be both strained and released. The mechanism also secures that the torque is held in the torsion spring once a dose is set and that the torque is releasable during dose injection.

The torsion spring engine is a separate unit which is secured in a housing 10 to thereby form an injection device. The torsion spring engine is proximally closed by a spring base 20. The torsion spring engine drives a piston rod 75 which is engaged by a nut member 30. Distally to the nut member 30, the housing 10 usually carries a not-shown cartridge-holder which secures a cartridge containing the liquid drug to be injected by forward movement of the piston rod 75. The cartridge-holder can either be permanently secured to the housing 10 in which case the injection device is a so-called pre-filled injection device or the cartridge holder can be removable secured to the housing 10 allowing a user to change the cartridge thus making the injection device durable.

The torsion spring engine disclosed in FIG. 1 henceforth comprises:
The spring base 20, (either being separate or a part of the housing structure).
The ratchet element 40 (also referred to as the dose setting element).
The clutch 60.
The drive tube 50.
The torsion spring S1.
The compression spring S2

The spring engine comprising at least these elements has the ability to strain the torsion spring S1 by rotation of the dose dial 15 and to release the torque applied to the torsion spring S1 by axial movement of the clutch 60 as will be explained in details in the following.

Both the spring base 20 and the nut member 30 are inrotatable secured to the housing 10 such that these three parts 10, 20, 30 together form one constructional element. In an alternative, the spring base 20 and the nut member 30 could each or both be moulded as integral parts of the housing 10. Further, the housing 10 could be a part of structure and is generally meant to be the outer shell surrounding the mechanism of the torsion spring driven injection device.

The dose setting arrangement comprises the dose dial 15, the ratchet element 40, the drive tube 50 and the clutch 60 as these elements rotate together during dose setting as will be explained. The dose dial 15 is coupled to the ratchet element 40 such that the ratchet element 40 rotates together with the dose dial 15 during dose setting and during dose adjustment.

To drive the injection a torsion spring S1 is at its distal end secured to the drive tube 50 and at its proximal end to the spring base 20.

The drive tube 50 is on the outer surface provided with a number of outwardly pointing flanges 52 as seen in FIG. 1 which slides in tracks 65 provided on the inner surface of the clutch 60 as depicted in FIG. 2. Further, teeth 51 also provided on the outer surface of the drive tube 50 slides in longitudinal openings 61 provided in the clutch 60. The clutch 60 is thus able to slide proximally in relation to the drive tube 50 a distance indicated by the arrow “C” in FIG. 2. However, in all situations are the clutch 60 and the drive tube 50 rotational bound to rotate together in unison.

A compression spring S2 is provided between the drive tube 50 and the clutch 60 urging the clutch 60 in the distal direction and urging the drive tube 50 in the proximal direction. As seen in FIG. 2, the compression spring S2 abuts the clutch 60 on a shelve provided internally in the clutch 60.

The inner surface of the clutch 60 is distally provided with a plurality of inwardly pointing teeth 62 which engages similar outwardly pointing teeth 71 provided on a piston rod guide 70.

The inwardly pointing teeth 62 on the inside of the clutch 60 however only engages the outwardly pointing teeth 71 on the piston rod guide 70 when the clutch 60 is moved to its proximal position against the bias of the compression spring S2 as will be explained.

The piston rod guide 70 is further provided with a non-circular inner surface 72 for engaging a piston rod 75 which on the outer surface is provided with a helical thread 76 which engages a similar internal thread 31 provided in the nut member 30.

The piston rod 75 is further formed with a non-circular cross section 77 which in the disclosed embodiment is formed as a number of longitudinal flat recesses. However, the non-circular cross section 77 can be made in many different ways e.g. as a longitudinal track. The purpose of the non-circular cross section 77 is to engage with the non-circular inner surface 72 of the piston rod guide 70 such that rotation of the piston rod guide 70 is directly transformed to a rotation of the piston rod 75.

Whenever the piston rod 75 rotates it is moved both rotational and axially (i.e. helically) due to the engagement between the outer thread 76 on the piston rod 75 and the internal thread 31 in the nut member 30.

In a different and not-shown embodiment the internal thread 31 can be provided in the piston rod guide 70 and the non-circular inner surface 72 can be provided in the nut member 20. In such embodiment, the piston rod 75 would move axially forward without any rotation when the piston rod guide 70 is rotated relatively to the nut member 20.

In order to visually inspect the size of the individual dose being set by rotating the dose dial 15, a scale drum 35 is provided. This scale drum 35 is on the outer surface provided with indicia 36 which are visible through a window 11 in the housing 10. The outer surface is further provided with a helical thread 37 which engage a similar thread or thread segment provided on the inside surface of the housing 10 such that the scale drum 35 moves helically in relation to the housing 10 when rotated.

On the inner surface the scale drum 25 is provided with a longitudinal track 38 which engages a similar protrusion 63 provided on the outer surface of the clutch 60 such that the scale drum 35 is able to rotate together with the clutch 60 but is axial slidable in relation to the clutch 60. A second protrusion also engaging the track 38 on the scale drum 35 can as depicted in FIG. 2 be provided distally on the clutch 60 to balance the movement of the scale drum 35.

The clutch 60 is further provided with an internal toothing 64 on the inside which internal toothing 64 engages outwardly pointing teeth 41 provided on the ratchet element 40 such that the clutch 60 follows rotation of the ratchet element 40 when the clutch 60 is in its proximal position.

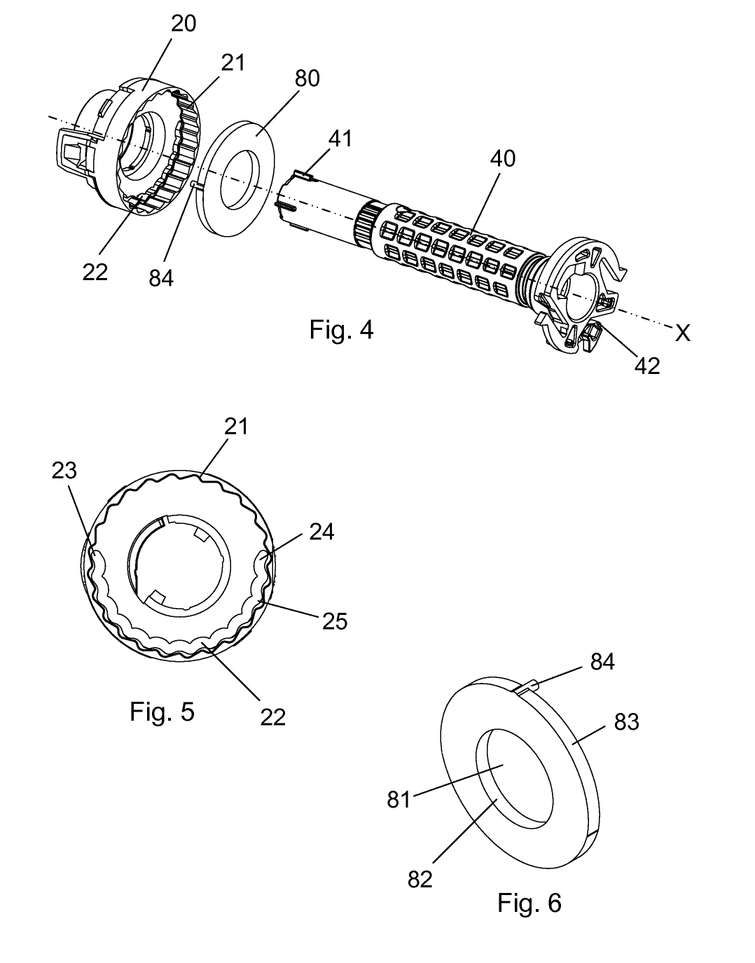

The ratchet element 40 is rotated by the dose dial 15 via a set of transfer arms 44 provided on the ratchet element 40 and engaged by the dose dial 15 to transfer rotation to the ratchet element 40. The ratchet element 40 is further provided with a radial tooth 42 which engages a toothed ring 21 (see e.g. FIG. 4) provided proximally in the spring base 20 such that the ratchet element 40 can only be rotated in one direction in relation to the spring base 40. The allowed direction that the ratchet element 40 can be rotated by the dose dial 15 is the one that sets and increases the dose size.

However, the dose dial 15 is further provided with means for moving the radial tooth 42 out of its engagement with the toothed ring 21 of the spring base 40 which allows the dose dial 15 to be rotated in the opposite direction to decrease a set dose size.

The dial down rotation of the ratchet element 40 is primarily done by the torque stored in the torsion spring S1 which is released when the radial tooth 42 is moved radially towards the centre line “X”. If the user does not continue rotating the dose dial 15 in the dose lowering direction, the radial tooth 42 will be caught in the previous valley of the toothed ring 21 such that the set dose is lowered with one single increment.

When setting a dose to be ejected, the user rotates the dose dial 15 which in unison rotates the ratchet element 40 due to the engagement with the transfer arms 44. Since the ratchet element 40 engages the clutch 60 via the outwardly pointing teeth 41 engaging the toothing 64, the clutch 40 follows the rotation of the ratchet element 40 and thus of the dose dial 15. The scale drum 35 follows the rotation of the clutch 60 and moves helically due to its engagement with the housing 10 thus allowing the user to visually see the size of the dose being set.

The clutch 60 further rotational engages the drive tube 50 which is therefore also rotated. This rotation strains the torsion spring S1 which is encompassed between the drive tube 50 and the spring base 20. The torque so build in the torsion spring S1 is secured by the releasable one-way engagement between the radial teeth 42 of the ratchet element 40 and the toothed ring 21 of the spring base 20.

The toothed ring 21 is formed such that it prevents relative rotation of the radial teeth 42 in the not dose setting direction. However, as mentioned, the radial teeth 42 can be forced radially out of its engagement with the toothed ring 21 allowing the torsion spring S1 to counter rotate the ratchet element 40 via its engagement with the clutch 60 to lower a set dose when the dose dial 15 is rotated in the not dose setting direction.

When not injecting e.g. during dose setting, the clutch 40 is urged to its distal position by the compression spring S2. In this position the teeth 62 are disengaged from the external teeth 71 on the piston rod guide 70 as seen in FIG. 2. The piston rod guide 70 is thus free to rotate when the clutch 60 is in its distal position.

When the torsion spring engine herein described is used in a prefilled injection device the piston rod 75 would abut a plunger (e.g. via a piston rod foot) provided inside the cartridge containing the liquid drug to be injected. The fact that the piston rod guide 70 is freely rotatable between injections allows the piston rod 75 to move axially should the liquid drug in the cartridge move the plunger as a consequence of a temperature change.

The torque applied to the torsion spring S1 during rotation of the dose dial 15, the ratchet element 40, the drive tube 50 and the clutch 60 is thus held by the engagement between the internal toothing 64 on the clutch 40 and the outwardly pointing teeth 41 on the ratchet element 40 and between the radial tooth 42 on the ratchet element 40 and the toothed ring 21 of the spring base 20.

In order to eject the set dose, the clutch 60 is moved in the proximal direction as indicated by the arrow “C” in FIG. 2. Once the internal toothing 64 on the clutch 60 is disengaged from the outwardly pointing teeth 41 on the ratchet element 40 nothing holds the clutch 60 and the torsion spring S1 will rotate the drive tube 50 and the clutch 60 in unison. The ratchet element 40 which no longer is coupled to the clutch 60 will remain in its rotational position. At the same time as the clutch 60 is moved proximally, the inwardly pointing teeth 62 on the clutch 60 engages the external teeth 71 on the piston rod guide 70 which therefore rotates together with clutch 60 and the drive tube 50 during ejection.

The clutch 60 is preferably moved in the proximal direction by a needle shield abutting the skin of the user during injection, however, the housing could alternatively be provided with a slidable button which could be operated by the user to push the clutch 60 in the proximal direction.

Rotation of the piston rod guide 70 is directly transformed to a rotation of the piston rod 75 which thereby is screwed forward in the internal thread 31 of the nut member 30 such that the plunger inside the attached cartridge is moved in the distal direction to press out liquid drug from the cartridge.

### The End-of-Content Mechanism, Example 1

In order to keep track of the number of set an ejected doses an End-of-Content system is disclosed in a first embodiment in the FIGS. 1 to 6, which EoC system comprises the ratchet element 40, the spring base 20 and a cycloid disc-shaped EoC element 80 as disclosed in FIG. 4.

The ratchet element 40 which is disclosed in details in FIG. 3 is rotated during dose setting around the centre axis X of the injection device as indicated in FIG. 4. However, the ratchet element 40 is provided with an annular bearing flange 43 which is formed eccentric such that the axis Y of the annular bearing flange 43 is offset a small distance from the centre axis X as seen in FIG. 3.

The cycloid disc-shaped EoC element 80 has, as best seen in FIG. 4 and in FIG. 6, a circular centre opening 81 defining an inner periphery 82 and an outer periphery 83. The inner periphery 82 rolls on the annular flange 43 of the ratchet element 40 during dose setting and the outer periphery rolls 83 on the toothed ring 21 of the spring base 20 and can be formed with one or more external teeth engaging the toothed ring 21.

The relationship between the diameter of the outer periphery 83 on the cycloid disc-shaped EoC element 80 and the inner diameter of the toothed ring 21 together with the small distance between the centre axis X of the ratchet element 40 and the centre line Y of the annular flange 43 and thus the cycloid disc-shaped EoC element 80 is such that the cycloid disc-shaped element 80 is rotated only a short distance for each full rotation of the annular flange 43 on the ratchet element 40. The rotational direction of the cycloid disc-shaped EoC element 80 is opposite the rotational direction of the ratchet element 40.

This hypocycloid working principle of this End-of-Content system is explained in details in WO 2014/117944 which is hereby incorporated by reference.

As can be best seen in FIG. 4 and in FIG. 6, the cycloid disc-shaped EoC element 80 is on its distal surface provided with an axially extending protrusion 84. This axially extending protrusion 84 engages a track 22 provided in the spring base 20. The track 22 is preferably formed with a number of valleys 25. In the depicted example in FIG. 4 and FIG. 5, the track 22 consists of 14 such valleys 25. A start valley 23 rotationally followed by 12 ordinary valleys 25 and at the end a stop valley 24.

The axially extending protrusion 84 can as disclosed in FIG. 6 also form an external teeth engaging the toothed ring 21.

When delivering the injection device to the user, the axially extending protrusion 84 is located in the start valley 23 thus leaving 12 valleys 25 free to travel before entering the stop valley 24. Further the diameter relation between the outer periphery 83 of the cycloid ring 80 and the inner diameter of the toothed ring 21 of the spring base 20 is selected such that the axially extending protrusion 84 moves through one valley 25 for each full rotation of the ratchet element 40. The axially extending protrusion 84 is thus able to move through the remaining 12 valleys 23 before coming to the stop valley 24 of the track 22. When the axially extending protrusion 84 is located in the stop valley 24 it is not possible to rotate the Cycloid disc-shaped EoC element 80 further which also prevents the ratchet element 40 from rotating further.

Since the ratchet element 40 follows the rotational movement of the dose dial 15, this design allows the dose dial 15 to rotate 12 full rotations before the axially extending protrusion 84 enters the stop valley 24 and thus abuts the end wall of the track 22. Once this happen the cycloid disc-shaped EoC element 80 is prevented from further rotation. This also means that neither the ratchet element 40 nor the dose dial 15 can rotate further in the dose setting direction. However, it is still possible to rotate the dose dial 15 in the opposite direction which moves the axially extending protrusion 84 in the opposite direction and thus lowers the size of the set dose.

If the herein disclosed EoC system is used for a torsion spring driven injection device for injecting insulin such injection devices often carries 300 I.U. of insulin in the cartridge and has a dose setting of 24 I.U. for each full revolution of the dose dial 15. The result being that once the user has rotated the dose dial 15 a number of 12 full revolutions then (12×24) 288 I.U. has been set and ejected. In order to stop further rotation once exactly 300 I.U. has been set and ejected, the distance from the last ordinary valley 25 to the stop valley 24 needs to be equal to only half rotation of the dose dial 15. The Disc-shaped EoC element-shaped EoC element is thus able to perform 12.5 rotations thus allowing (12.5×24) 300 I.U. to be ejected. However, any number of valleys 25 can be selected depending on how many rotations should be allowed before the cycloid disc-shaped EoC element 80 comes to its stop position.

### The End-of-Content Mechanism, Example 2

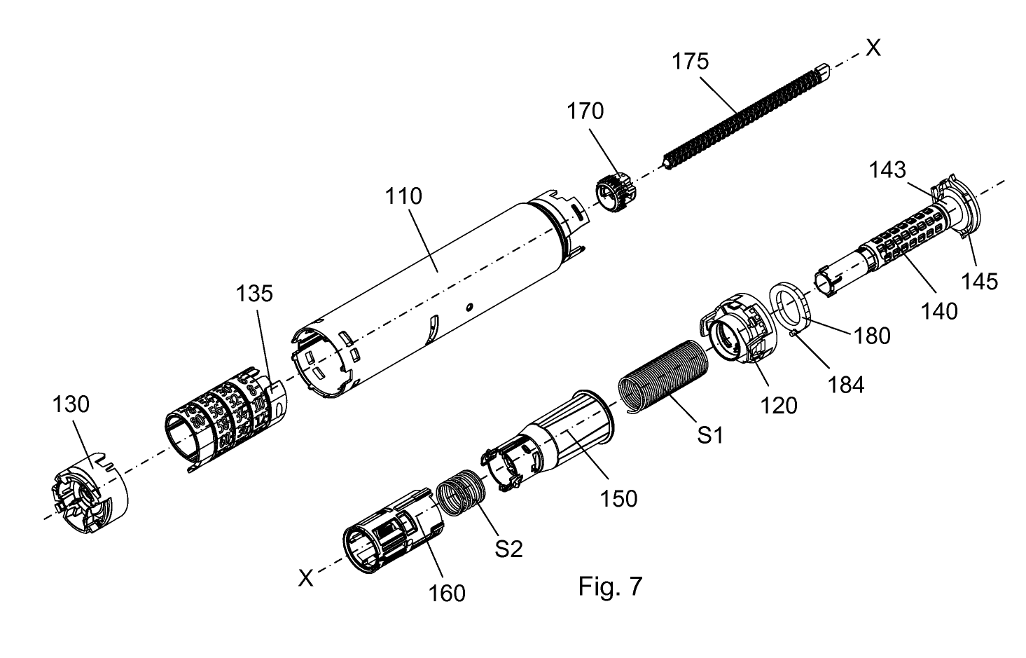

FIG. 7 to FIG. 12 disclose a second embodiment wherein the same constructional elements are numbered using the same reference numbers however with a “1” in front, the piston rod is henceforth numbered “175” in this embodiment.

The spring engine herein disclosed operates in the same way as the spring engine of the first embodiment; however, the EoC system is different as will be explained.

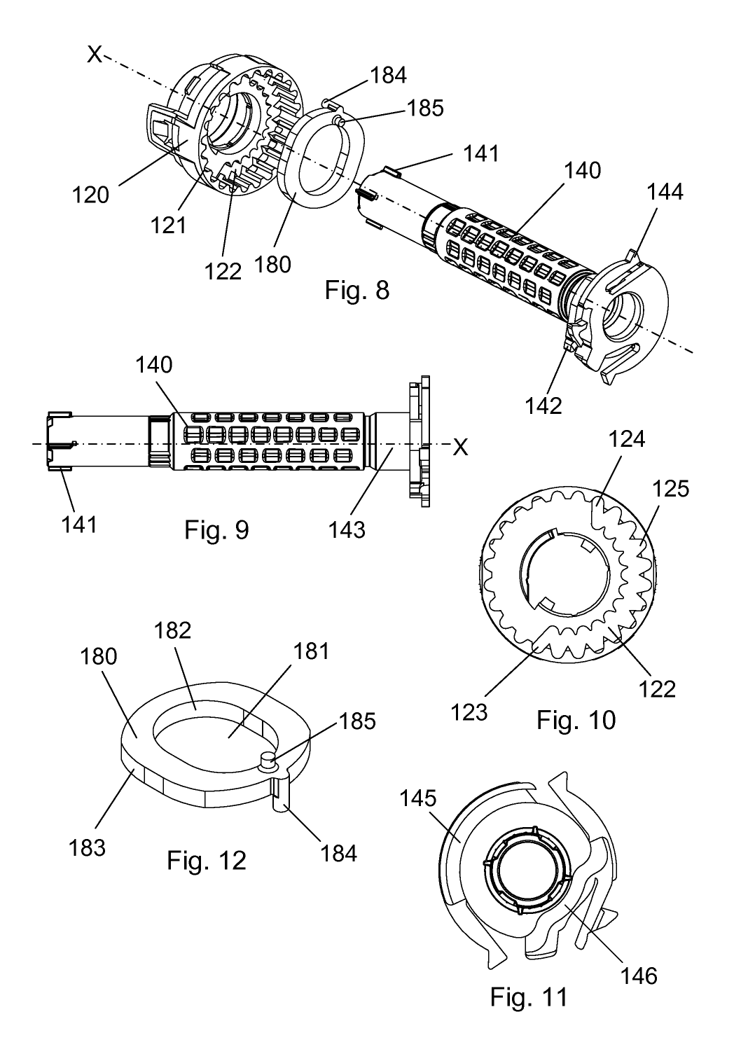

The EoC system primarily consist of three parts as disclosed in FIG. 8. These parts are; the ratchet element 140 (FIG. 9), the disc-shaped EoC element 180 (FIG. 12) and the spring base 120.

FIG. 12 discloses the cycloid disc-shaped EoC element 180 of the second embodiment in details. On the distal surface an axially extending first protrusion 184 is present as in the first embodiment. On the proximal surface a second protrusion 185 is provided which also extend axially. Further, the centre opening 181 has an oval shape such that the disc-shaped EoC element 180 is able to oscillate in a radial direction.

The first protrusion 184 is guided in a first track 122 provided in the spring base 120 as seen in FIG. 8 whereas the second protrusion 185 is guided in a second track 145 provided in the ratchet element 140. The spring base 120 is, as in the first embodiment, an operational part of the housing 110 either physically or by engagement. Internally the spring base 120 is provided with a toothed ring 121 supporting the outer periphery 183 of the disc-shaped EoC element 180 and a first track 122 as disclosed in FIG. 8.

The first track 122 is provided with a start valley 123, a stop valley 124 and a predetermined number of ordinary valleys 125 whereas the second track 145 is primarily a circular track but with a curled part 146 as best seen in FIG. 11.

The ratchet element 140 is, as in the first embodiment, provide with an annular flange 143 supporting the disc-shaped EoC element 180 as best seen in FIG. 9. However, in this embodiment, the annular flange 143 is circular allowing the opening 181 of the Disc-shaped EoC element-shaped EoC element 180 to rotate there upon.

As in the first embodiment, the rotational force is transmitted from the dose dial (not shown in this embodiment) to the ratchet element 140 via the transfer arms 144 and the radial tooth 142 engages the toothed ring 121 of the spring base 120.

In use, the user rotates the dose dial, which is not depicted in FIG. 7. This rotation is transformed to a rotation of the ratchet element 140 as in the first embodiment. At this moment, the first protrusion 184 is located in the start valley 123 and the second protrusion 185 is located in the circular part of the track 145. As long as the second protrusion 185 is located in the circular part of the second track 145, the disc-shaped EoC element 180 is not rotated when the ratchet element 140 is rotated.

At some point during the rotation of the ratchet element 140, the second protrusion 185 enters the curled part 146 of the second track 145. This will force the Disc-shaped EoC element-shaped EoC element 180 to oscillate in the radial direction such that the second protrusion 185 is moved toward the centre line X as best seen in FIG. 8. As the first protrusion 184 moves out of the curled track 146, the second protrusion 185 is moved away from the centre line X and thus into the first ordinary valley 125.

This pattern of movement will continue as the user sets subsequently doses and the second protrusion 185 will move one ordinary valley 125 for each full rotation of the ratchet element 140. The disc-shaped EoC element 180 will thus move in the same rotational direction as the ratchet element 140 and after a predetermined number of rotations of the ratchet element 140 and henceforth of the dose dial, the first protrusion 185 will enter the stop valley 124 of the first track 122 and prevent both the Disc-shaped EoC element-shaped EoC element 180 and the ratchet element 140 from further rotation.

### The End-of-Content Mechanism, Example 3

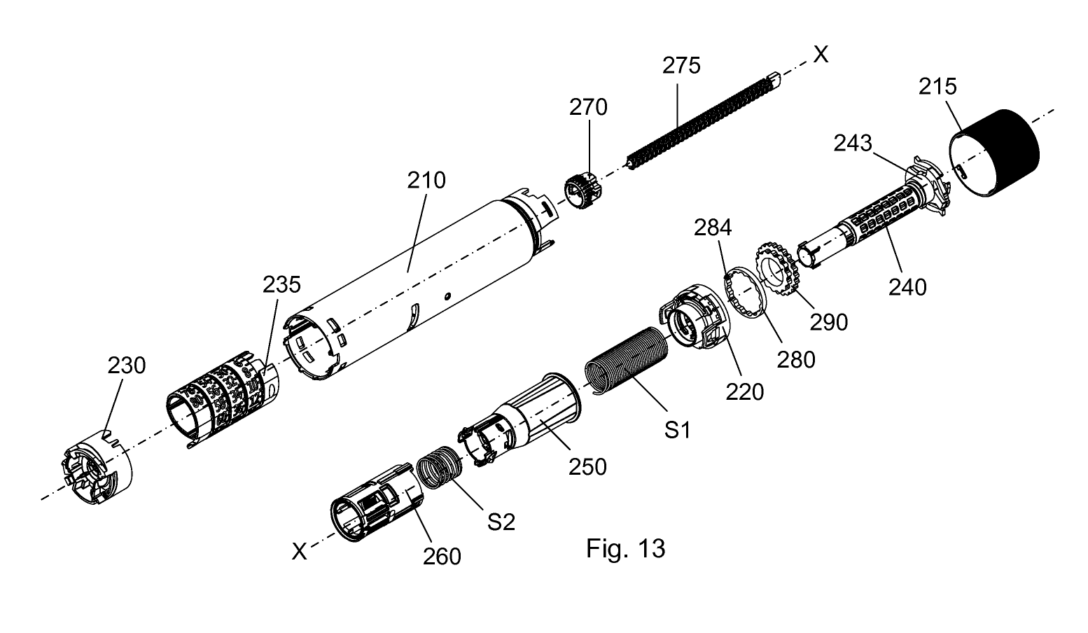

In the third embodiment disclosed in FIGS. 13 to 17b, the spring engine operates as in both the previous two embodiments. The housing 210 distally carries a nut member 230 and proximally a spring base 220. The torsion spring S1 is encompassed between the housing 210 and the drive tube 250. The release clutch 260 is coupled to the drive tube 250 and axially movable in relation to the drive tube 250. Between injections the release clutch 260 is urged in the distal direction by the compression spring S2 such that the release clutch 260 in this first position is decoupled from the piston rod guide 270 allowing the piston rod guide 270 to rotate independently of the release clutch 260. At the same time the release clutch 260 is coupled with the rotatable dose setting element 240 to rotate simultaneously with the rotatable dose setting element 240 in order to set a dose.

In the second position the release clutch 260 is decoupled from the rotatable dose setting element 240 such that the release clutch 260 is able to rotate independently of the rotatable dose setting element 240 under influence of the torsion spring S1. Further, in this second position the release clutch 260 is coupled with the piston rod guide 270 such that the piston rod guide 270 rotates simultaneously with the release clutch 260 in order to drive the piston rod 275 forward.

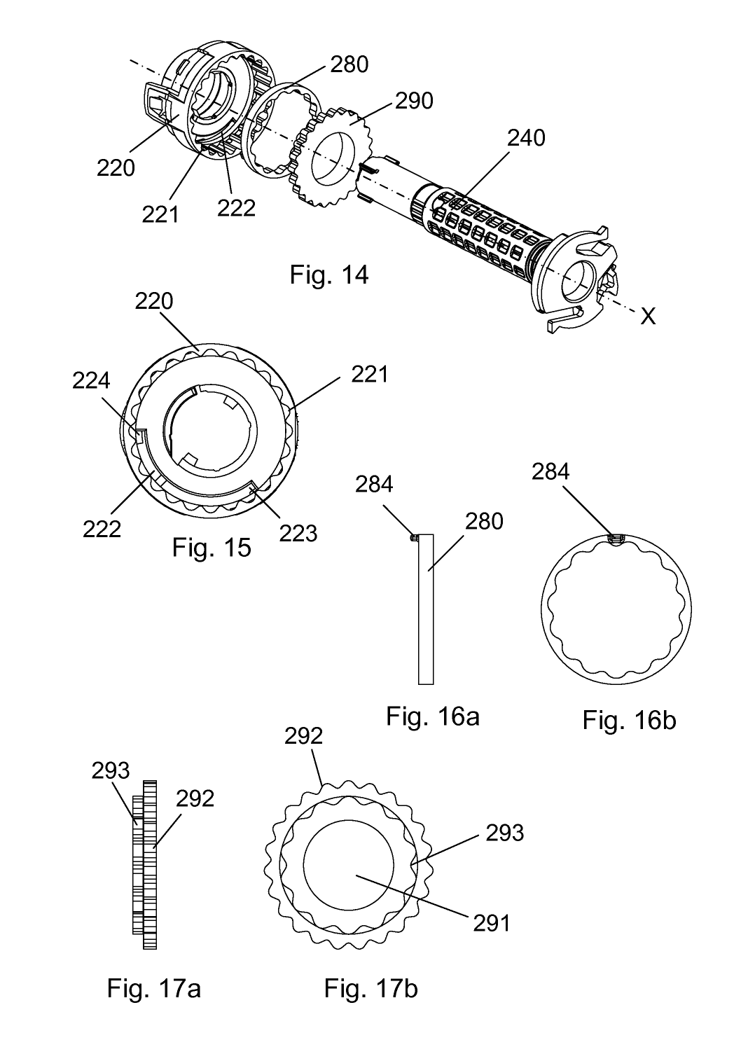

The EoC system is disclosed in FIG. 14 and comprises the ratchet element 240, the spring base 220 and the disc-shaped EoC element 280. In addition an extra cycloid ring-shaped EoC gear element 290 is used in this embodiment.

This cycloid EoC gear element 290 has a centre opening 291 which rolls on the annual flange 243 of the ratchet element 240. The annular flange 243 on the ratchet element 240 is in this embodiment eccentric as in the first embodiment thereby introducing a hypocycloid rotational movement to the cycloid ring-shaped EoC gear element 290 during rotation of the ratchet element 240. This is similar to the first embodiment and the cycloid EoC gear element 290 thus rotates in the opposite rotational in relation to the ratchet element 240.

Whenever the user rotates the ratchet element 240 one full revolution by rotating the dose dial 215, the cycloid ring-shaped EoC gear element 290 rotates a smaller angular distance in the opposite rotational direction.

As can be seen from the FIG. 17, the cycloid ring-shaped EoC gear element 290 has a circular opening 291 and a proximal toothed periphery 292 and a distal toothed periphery 293. The proximal toothed periphery 292 rolls on the toothed ring 221 of the spring base 220 in a hypocycloid movement whereas the distal toothed periphery 293 is coupled to the disc-shaped EoC element 280.

Rotation of the cycloid EoC gear element 290 thus introduces a rotation of the disc-shaped EoC element 280 in the opposite rotational direction of the cycloid EoC gear element 290. The result being that whenever the ratchet element 240 is rotated e.g. in a clockwise direction, the cycloid ring-shaped EoC gear element 290 rotate a smaller angle in the anti-clockwise direction which again forces the disc-shaped EoC element 280 to rotate an even smaller angle in the clockwise direction thus following the rotational direction of the ratchet element 240.

When delivering the torsion spring driven injection to the user the axially extending protrusion 284 of the disc-shaped EoC element 280 is located in the start position 223 of the track 222 formed in the spring base 220. Every time the ratchet element 240 is rotated one full rotation in one rotational direction, the axially extending protrusion 284 is moved a smaller angle in the same rotational direction in the track 222. The angular movement of 284 is determined by the gearing ratio and after a predetermined number of rotations of the ratchet element 240 the axially extending protrusion 284 comes to the end position 224 in the track 222 where the axially extending protrusion 284 abut the end of the track 222 and thereby prevents further dose setting.

Some preferred embodiments have been shown in the foregoing, but it should be stressed that the invention is not limited to these, but may be embodied in other ways within the subject matter defined in the following claims.

1. A torsion spring driven injection device for delivering individually set doses of a liquid drug, comprising:
a housing,
a rotatable dose setting element which is axially fixated in the housing,
a piston rod having an outer surface with an outer thread which extend helically in a longitudinal direction and which outer surface further is provided with a longitudinal extending engagement surface such that the outer surface of the piston rod has a non-circular shape,
a nut member having an inner thread mating the outer thread of the piston rod,
a piston rod guide engaging the longitudinal extending engagement surface of the non-circular cross section of the piston rod such that the piston rod is moved axially when the piston rod guide and the nut member are rotated relatively to each other, and wherein either the nut member or the piston rod guide is non-rotational engaged in the housing,
a drive tube axially fixated in the housing and engaging an axially movable dose release clutch,
a torsion spring which is strained by rotation of the rotatable dose setting element, and which torsion spring is encompassed between the housing and the drive tube, such that a torque is build up in the torsion spring during rotation of the drive tube relatively to the housing during dose setting and which torque is released to rotate the drive tube during ejection,
wherein the release clutch is axially movable in relation to the drive tube between a first position and a second position;
wherein the release clutch is decoupled from the piston rod guide such that the piston rod guide is able to rotate independently of the release clutch and the release clutch is coupled with the rotatable dose setting element to rotate simultaneously with the rotatable dose setting element in order to set a dose,
wherein the release clutch is decoupled from the rotatable dose setting element such that the release clutch is able to rotate independently of the rotatable dose setting element under influence of the torsion spring, and the release clutch is coupled with the piston rod guide such that the piston rod guide rotates simultaneously with the release clutch in order to drive the piston rod, and
wherein the release clutch is rotational locked to the drive tube such that the release clutch and the drive tube rotates in unison but can move axially in relation to each other,
wherein the release clutch and the drive tube rotate together in a first rotational direction by rotation of the dose setting element when the release clutch is in the first position thereby straining the torsion spring to build up torque, and
the release clutch and the drive tube rotate together in a second opposite rotational direction by the torsion spring when the release clutch is in the second position thereby releasing the torque of the torsion spring.

2. The torsion spring driven injection device according to claim 1 wherein, the nut member is non-rotational engaged in the housing.

3. The torsion spring driven injection device according to claim 1, wherein, the housing comprises a spring base to which the torsion spring is attached.

4. The torsion spring driven injection device according to claim 1, wherein a compression spring is provided between the drive tube and the release clutch urging the drive tube and the release clutch in opposite axial directions.

5. The torsion spring driven injection device according to claim 1, wherein the ratchet element is provided with an annular bearing flange rotatably carrying a rotatable disc-shaped End-of-Content (EoC) element.

6. The torsion spring driven injection device according to claim 5, wherein the annular bearing flange is eccentric.

7. The torsion spring driven injection device according to claim 5, wherein the disc-shaped EoC element is provided with one or more axially extending protrusions.

8. The torsion spring driven injection device according to claim 5, wherein the housing is provided with a peripheral track guiding at least one of the protrusions provided on the disc-shaped EoC element.

9. The torsion spring driven injection device according to claim 5, wherein an additional ring-shaped EoC gear element is provided.

10. The non-axial working End-of-Content mechanism for the torsion spring driven injection device according to claim 5, wherein:
the rotatable dose setting element is rotatable around a first axis (X), and
the disc-shaped EoC element is rotatable around a second axis (Y) which second axis is dislocated in relation to the first axis (X),
wherein arresting structure is provided for stopping the rotation of the cycloid disc in a predetermined position.

11. The non-axial working End-of-Content mechanism according to claim 10, wherein the arresting structure comprises an axially extending protrusion provided on the disc-shaped EoC element and a stopping valley provided in a guiding track in the housing.

12. The non-axial working End-of-Content mechanism for the torsion spring driven injection device according to claim 5, wherein:
the rotatable dose setting element is rotatable around a first axis (X), and
the disc-shaped EoC element has a first protrusion guided in a first track and a second protrusion guided in a second track,
wherein the second track is provided with at least one curled part introducing at least one oscillating radial movement to the disc-shaped EoC element for each full rotation of the dose setting element, and arresting structure for stopping the rotation of the disc-shaped EoC element in a predetermined position.

13. The non-axial working End-of-Content mechanism according to claim 12, wherein the first track is provided in the housing, and the second track is provided in the dose setting element.

14. The non-axial working End-of-Content mechanism for the torsion spring driven injection device according to claim 5, wherein:
the rotatable dose setting element is rotatable around a first axis (X), and
the ring-shaped EoC gear element is rotatable around a second axis (Y) which second axis is dislocated in relation to the first axis (X), whereby rotation of the ring-shaped EoC gear element is transformed to a rotation of the disc-shaped EoC element,
wherein arresting structure is provided for stopping the rotation of the disc-shaped EoC element in a predetermined position.

15. The non-axial working End-of-Content mechanism according to claim 14, wherein the arresting structure comprises an axially extending protrusion provided on the disc-shaped EoC element and a stopping position provided in a guiding track in the housing.

16. The torsion spring driven injection device according to claim 5, wherein the spring base is provided with a peripheral track guiding at least one of the protrusions provided on the disc-shaped EoC element.

17. The non-axial working End-of-Content mechanism according to claim 10, wherein the arresting means structure comprises an axially extending protrusion provided on the disc-shaped EoC element and a stopping valley provided in a guiding track in the in the spring base.

18. The non-axial working End-of-Content mechanism according to claim 12, wherein the first track is provided in the spring base, and the second track is provided in the dose setting element.

19. The non-axial working End-of-Content mechanism according to claim 14, wherein the arresting structure comprises an axially extending protrusion provided on the disc-shaped EoC element and a stopping position provided in a guiding track in the spring base.
